(19) United States
(12) Patent Application Publication
(10) Pub. No.: US 2009/0169599 A1
(43) Pub. Date: Jul. 2, 2009

(54) SCIENTIFICALLY MODULATED AND REPROGRAMMED TREATMENT (SMART) FAS/FASL VIRUS TECHNOLOGY INTENDED TO NEUTRALIZE T-HELPER CELLS INFECTED WITH THE HUMAN IMMUNODEFICIENCY VIRUS

(76) Inventors: Lane Bernard Scheiber, Annandale, VA (US); Lane Bernard Scheiber, II, Grosse Ile, MI (US)

Correspondence Address: LANE B. SCHEIBER, 7864 OAK RIVER DRIVE, GROSS ILE, MI, 48138, US

(21) Appl. No.: 11/968,007

(22) Filed: Dec. 31, 2007

(51) Int. Cl. A61K 9/70; A61K 9/48

(52) U.S. Cl. 424/444; 424/463

(57) ABSTRACT

Scientifically Modulated And Reprogrammed Treatment (SMART) Virus Fas/FasL technology is intended to terminate T-Helper cells infected with the Human Immunodeficiency Virus. The SMART-Fas/FasL Virus carrying Fas and FasL cell-surface receptors is capable of engaging a T-Helper cell infected by HIV that is expressing one or more FasL cell-surface receptors. When a T-Helper cell infected with HIV encounters a SMART-Fas/FasL Virus, the infected T-Helper cell's FasL cell-surface receptor will engage the SMART-Fas/FasL Virus's Fas receptor, then the SMART-Fas/FasL Virus's FasL will engage the infected T-Helper cell's Fas receptor, which will initiate apoptosis in the infected T-Helper cell. Given the HIV infected T-Helper cell will be triggered to die, HIV's safe haven inside the T-Helper cell will be eliminated and the threat of Acquired Immunodeficiency Syndrome caused by HIV is averted.

### CROSS-REFERENCE TO RELATED APPLICATIONS

None.

### STATEMENT REGARDING SPONSORED RESEARCH OR DEVELOPEMNT

Not applicable.

### REFERENCE TO SEQUENCE LISTING, A TABLE, OR COMPUTER LISTING COMPACT DISC APPENDIX

Not applicable.

©2008 Lane B. Scheiber and Lane B. Scheiber II. A portion of the disclosure of this patent document contains material which is subject to copyright protection. The copyright owners have no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

1. Field of the Invention

This invention relates to any medical device intended to physically interact directly with T-Helper cells infected with the Human Immunodeficiency Virus (HIV) or infected with other virus, to neutralize the infectious threat of the virus.

2. Description of the Background Art

The Human Immunodeficiency Virus (HIV), which is responsible for Acquired Immunodeficiency Disease Syndrome (AIDS), threatens the lives of an estimated 170 millions of people worldwide. There are different strains of HIV that exist around the world. Most predominantly HIV-1 exists worldwide and HIV-2 is generally found in Western Africa, the western coastal regions of India and in Europe. Amongst HIV-1 and HIV-2 they can be further subdivided into different strains including an ‘R5’ strain which uses a CCR5 cell-surface receptor on a T-Helper cell to identify and access its host and an ‘X4’ strain which uses a CXCR4 cell-surface receptor located on a T-Helper cell to identify and access its host. The approach to controlling the disease caused by HIV has been the application of drugs directed at interfering with the replication process, in an attempt to slow down the rate of replication of the virus. Millions of people continue to die and the virus continues to pose an escalating worldwide threat despite current treatment strategies. The virus is generally communicated between individuals by contact with body fluids carrying intact HIV.

Though there are recognized differences between HIV-1 and HIV-2, for purposes of further discussion the term ‘HIV’ will refer to both HIV-1 and HIV-2, unless otherwise noted. HIV is a retrovirus with its genetic material in the form of two identical copies of a positive sense single stranded ribonucleic acid (RNA) molecule, each approximately 9500 nucleotides long. HIV is approximately 50 to 150 nm in diameter, about one seventieth the size of a white cell carrying the marker Cluster Designation 4 (CD4) exterior cell-surface receptor.

A eukaryote refers to a nucleated cell. Eukaryotes comprise nearly all plant and animal cells. Animal cells generally are comprised of a cell membrane, cytoplasm, a nucleus and organelles. The cell membrane consists of a lipid bilayer where two layers of lipid molecules oriented with their polar ends pointed outside of the membrane and their nonpolar ends points toward the inside of the membrane. Polarized ends of the lipid molecules are hydrophobic, therefore the lipid bilayer functions to control the movement of water, nutrients and hormones in and out of a cell. A variety of receptors affixed to the exterior of the lipid bilayer membrane assist in a cell communicating with its environment. The cytoplasm inside a cell, which forms the interior fluid matrix of the cell, is comprised of amino acids and nutrients. The nucleus is surrounded by a double membrane (often referred to as a nuclear membrane) and contains the majority of a cell's genetic material. Organelles are structures generally found in the cytoplasm that perform specialized functions of cells. Organelles found inside a cell may include the mitochondria, endoplasmic reticulum, Golgi complex, lysosomes, vacuoles.

Genetic material in a eukaryote is generally in the form of deoxyribonucleic acid (DNA) with the majority located in the nucleus of the cell, but DNA may also be found in the mitochondria of cells. By the process of transcription, a section of the DNA is read by a polymerase and a molecule of ribonucleic acid (RNA) is generated. DNA is comprised of sections of combinations of four nucleotides: adenine, cytosine, guanine, and thymine. When two strands of nucleotides are arranged together, such as in the double helix configuration of chromosomal DNA, adenine on one strand is always matched to thymine in the opposing strand, and cytosine on one strand is always matched to guanine in the opposing strand. RNAs generated by polymerases reading nuclear DNA are usually single stranded chains of nucleotides, constructed of similar adenine, cytosine and guanine nucleotides as DNA, but instead of ‘thymine’, RNAs are constructed with the nucleotide ‘uracil’. RNAs are generally divided into three categories including messenger RNA (mRNA), ribosomal RNA (rRNA) and transfer RNA (tRNA). Messenger RNAs are considered positive sense and interact with ribosomes to generate protein molecules. Ribosomes read the code physically built into the messenger RNAs, and with the aid of rRNAs and tRNAs, generate protein molecules by bonding together amino acids in linear configurations as directed by the code on a messenger RNA.

Blood cells are generally referred to as white blood cells and red blood cells. Thrombocytes, otherwise known as platelets, are flat disk-shaped cell fragments that circulate the blood to assist with clotting when required. White blood cells, also referred to as leukocytes, play an active role in the body's immune system. White blood cells are further divided into T-Cells and B-Cells. T-Helper cells, also known as CD4 T-lymphocytes or CD4 T-Cells, are a subset of white blood cells. T-Helper cells act to coordinate the body's immune response against infectious agents. A significant decline in the number of circulating T-Helper cells represents a state where the body is vulnerable to opportunistic infections such a pneumonia, fungal infections or other common ailments.

Viruses are obligate intracellular parasites designed to infect cells often with great specificity to a particular cell type it uses as a host. Virion is a term that refers to a complete structure of a virus as it exists outside of a host cell. Viruses do not carry out any biologically active processes on their own when outside a host cell. A virus requires a host in order to reproduce itself. Viruses circulate the environment without the need for nutrition or energy production through respiration. Viruses are in essence a vehicle that carries the genetic programming instructions necessary to cause an appropriate host cell to generate identical copies of the same virus. Some viruses, such as HIV, do introduce to their host cells programming instructions that result in toxic effects to the body as a whole.

HIV is considered to be approximately spherical in shape and comprised of an outer lipid bilayer envelope, a matrix protein, a capsid, two strands of RNA, nucleocapsid protein and proteins to assist in the replication process. The virus's core or capsid is icosahedral in shape and acts as a protective shell to carry the genetic payload. The capsid is comprised of numerous copies of the capsid protein (p24), the number and arrangement of the capsid proteins determines the overall dimensions of the capsid shell; HIV uses approximately 2,000 capsid proteins (p24) to construct its capsid. The capsid carries the two single strands of RNA each containing a copy of the virus's nine genes, the nucleocapsid protein, reverse transcriptase, protease and integrase. The nucleocapsid protein causes the RNA to coil up so that it can fit inside the capsid. The protein matrix consisting of protein 17 (p17) covers the capsid. The HIV envelope is derived from the plasma membrane of the host cell as the virus buds or pushes through the host cell's plasma membrane as it exits and migrates from the host cell. Anchored in and projecting out from the HIV's lipid bilayer outer membrane, otherwise referred to as an envelope, are exterior probes well known to the medical and scientific community as glycoprotein 120 (gp 120) and glycoprotein 41 (gp 41). The term glycoprotein refers to a protein with a carbohydrate attached. The gp 41 probe is anchored to the outer envelope and is in close proximity to the gp 120 probe. The probes can be found arranged together into protein complexes, which may contain up to three gp 120 probes and three gp 41 probes. Protein complexes have been described as ‘spikes’. It has been reported that an HIV outer envelope may project from ten to seventy-two said spikes.

A HIV virion transits the environment at large with its surface probes seeking to engage a human T-Helper cell. Human T-Helper cells express a number of cell-surface receptors on their outer plasma membrane including Cluster Designation 4 (CD4), Chemotactic Chemokine Receptor 5 (CCR5) and CX Chemokine Receptor 4 (CXCR4). HIV utilizes the human T-Helper cell, also known as a CD4 T-lymphocyte or CD4 T-Cell, as its host for the purpose of replicating copies of itself. To initiate its reproductive-cycle, the gp 120 probe on a HIV virion makes initial contact with a T-Helper cell's CD4 cell-surface receptor. Following the engagement of the gp 120 probe with the CD4 cell-surface receptor, the gp 120 probe alters its configuration to allow the HIV gp 41 probe to engage a second receptor on the surface of the T-Helper cell, either a CCR5 cell-surface receptor or a CXCR4 cell-surface receptor. Once the HIV virion's gp 120 probe has successfully engaged a T-Helper cell's CD4 cell-surface receptor and the HIV's gp 41 probe has successfully engaged the T-Helper cell's CCR5 or CXCR4 cell-surface receptor, then the HIV virion is able to transfer its capsid containing the two strands of ribonucleic acid (RNA) and the support proteins including reverse transcriptase, protease, and integrase into the T-Helper cell. Once the capsid has gained access to the interior of the T-Helper cell, utilizing the transferred HIV enzyme ‘reverse transcriptase’, the RNA molecules undergo reverse transcription to deoxyribonucleic acid (DNA). Protease helps modify HIV's genome. Aided by the integrase, the virus's RNA that has been transcribed into DNA migrates to the T-Helper cell's nucleus and is known to become inserted into the T-Helper cell's native DNA. The HIV genetic material then redirects the resources of the T-Helper cell to facilitate the manufacture of copies of HIV.

Most predominantly HIV-1 exists worldwide and HIV-2 is generally found in Western Africa, the western coastal regions of India and Europe. Amongst HIV-1 and HIV-2 that use CD4 as the initial cell-surface receptor to gain entry into a T-Helper cell, they can be further divided by an ‘R5’ strain which uses a CCR5 cell-surface receptor on a T-Helper cell to identify its host; an ‘X4’ strain which uses a CXCR4 cell-surface receptor located on a T-Helper cell to identify its host. It is also believed at least one strain of HIV-2 may infect a T-Helper cell without engaging a CD4 cell-surface receptor, but uses either a CCR5 or a CXCR4 cell-surface receptor on a T-Helper cell host. There has also been identified at least one strain of HIV-2 believed not utilize the CD4, CCR5 or the CXCR4 cell-surface receptors to engage a T-Helper cell host, the mode of entry utilized by this form or HIV virion is unknown at this time.

The Fas cell-surface receptor (also referred to as CD95 cell-surface receptor) appears naturally on the surface of T-Helper cells. The Fas cell-surface receptor, when triggered, will transmit into the cell a biologic signal to activate the process of apoptosis. Apoptosis is a natural process utilized to terminate a cell, resulting in cell death.

Naturally occurring T-Helper cells, not infected with HIV, help orchestrate the human body's immune response against infectious agents that threaten the health and integrity of the body. The HIV virus, by taking control and altering the function of the T-Helper cells in the body, creates a state of ill health. By redirecting the T-Helper cell's function to produce copies of the HIV virus rather than coordinate appropriate immune responses against potentially infectious agents leaves the body as a whole vulnerable to attack by other infectious agents that can do harm to the tissues of the body. In addition, the HIV genome carries a ‘nef’ gene. Once the HIV's DNA is inserted into the host cell's native DNA, the nef gene provides instructions for a Fas ligand (FasL) cell-surface marker to be manufactured and expressed on the surface of the infected T-Helper cell. Noninfected T-Helper cells (meaning not infected with HIV) express a Fas cell-surface marker. When a HIV infected T-Helper cell expressing the Fas ligand (FasL) cell-surface marker encounters a Fas cell-surface marker on a noninfected T-Helper cell, a lethal biologic signal is transmitted to the noninfected T-Helper cell. That is when the FasL cell-surface marker engages a Fas cell-surface marker, the process of apoptosis is triggered in the noninfected T-Helper cell. By triggering apoptosis in noninfected T-Helper cells, HIV infected T-Helper cells are capable of killing the noninfected T-Helper cells they encounter. The clinical ramifications of Acquired Immunodeficiency Syndrome (AIDS) occur when the number of noninfected T-Helper cells declines to the point the immune system is unable to defend the body as a whole from dangerous infectious agents that would attempt to invade the body's tissues.

T-Helper cells infected with HIV pose possibly the greatest threat to the integrity of a body's immune system by terminating noninfected T-Helper cells, which a critical number of noninfected T-Helper cells is needed to defend the body from infectious agents. Since the clinical characteristics of AIDS appears to coincide more with the decline of non-infected T-Helper cells below a critical number and not necessarily with the number of HIV infected T-Helper cells, controlling the population of HIV infected T-Helper cells or ridding the environment of HIV infected T-Helper cells would be a successful approach to managing AIDS.

### BRIEF SUMMARY OF THE INVENTION

A Scientifically Modulated And Reprogrammed Treatment (SMART) Fas/FasL Virus is comprised of an inner capsid similar to the naturally occurring HIV icosahedral capsid. The SMART-Fas/FasL Virus capsid is encapsulated with a protein matrix similar to the protein matrix that encapsulates the HIV's capsid. A lipid bilayer envelope then encapsulates the matrix protein coat and capsid similar to the lipid bilayer that encapsulates the matrix protein of HIV. Two cell-surface receptors Fas and Fas ligand (FasL) would be fixed to the surface of the SMART-Fas/FasL Virus. The capsid of the medically therapeutic version of the SMART-Fas/FasL Virus would carry either no genetic payload or would carry RNA molecules that would not be capable of replicating the virus-like structure in the natural environment or causing any disease in any form of life. The amount of genetic payload or filler would relate to the intended size and stability of the SMART-Fas/FasL Virus. SMART-Fas/FasL Viruses are intended to engage T-Helper cells infected with HIV that are expressing the FasL cell-surface receptor. Once an infected T-Helper cell's FasL cell-surface receptor engages a Fas cell-surface receptor on the SMART-Fas/FasL Virus, the SMART-Fas/FasL Virus's FasL cell-surface receptor engages a Fas cell-surface receptor on the infected T-Helper cell. Once the infected T-Helper cell's Fas cell-surface receptor has been engaged, a biologic signal is triggered inside the infected T-Helper cell that initiates the process of apoptosis. The process of apoptosis terminates the infected T-Helper cell.

### DETAILED DESCRIPTION

Scientifically Modulated And Reprogrammed Treatment (SMART) Fas/FasL Virus technology is intended to neutralize T-Helper cells infected with the Human Immunodeficiency Virus. A Scientifically Modulated And Reprogrammed Treatment (SMART) Fas/FasL Virus is comprised of an inner capsid similar to the naturally occurring HIV icosahedral capsid. The SMART-Fas/FasL Virus capsid would be encapsulated with a protein matrix similar to the protein matrix that encapsulates the HIV's capsid. A lipid bilayer envelope then encapsulates the matrix protein coat and capsid similar to the lipid bilayer that encapsulates HIV's matrix protein and inner capsid. Two cell-surface receptors Fas (also known as CD98) and Fas ligand (FasL) would be fixed to the surface of the SMART-Fas/FasL Virus. The capsid of the therapeutic version of the SMART-Fas/FasL Virus would carry either no genetic payload or would carry RNA molecules that would not be capable of replicating the virus-like structure in the natural environment or causing any disease in any form of life. The amount of genetic payload or filler would relate to the intended stability of the SMART-Fas/FasL Virus. Within the SMART-Fas/FasL Virus's outer envelope are matrix protein and a capsid to act as the interior conformational structure to provide the appropriate size and shape to the outer envelope as needed for the intended use. The size of the SMART-Fas/FasL Virus ranges from 7 nanometer (nm) in thickness (the diameter of HIV is approximately 50 to 150 nm) to a diameter in the order of the size of a naturally occurring noninfected T-Helper cell, and up to a diameter of one meter. T-Helper cells are mobile structures and constantly alter their shape and size, but are approximately 3500 nm in diameter. The range in size of the SMART-Fas/FasL Virus is dependent upon the type of application for which the SMART-Fas/FasL Virus is intended to be utilized.

A SMART-Fas/FasL Virus is intended to engage a T-Helper cell infected with HIV that is expressing the FasL cell-surface receptor. Once an infected T-Helper cell's FasL cell-surface receptor engages a Fas cell-surface receptor on the said SMART-Fas/FasL Virus, the SMART-Fas/FasL Virus's FasL cell-surface receptor engages a Fas cell-surface receptor on the infected T-Helper cell. Once the infected T-Helper cell's Fas cell-surface receptor has been engaged, a biologic signal is triggered inside the infected T-Helper cell that initiates the process of apoptosis. Apoptosis is a naturally occurring biologic process present in cells that when initiated results in cell death. Activation of apoptosis terminates the infected T-Helper cell. In the event HIV is unable to successfully create additional copies of itself and unable to further threaten noninfected T-Helper cells, the threat of Acquired Immunodeficiency Disease Syndrome is successfully neutralized.

SMART-Fas/FasL Virus technology can be used as a cleaning device to neutralize and rid a surface or a fluid environment of HIV infected T-Helper cells. SMART-Fas/FasL Virus is intended to engage HIV infected T-Helper cells where ever it may exist.

HIV utilizes T-Helper cells as a natural factory for generating copies of HIV. HIV utilizes a fusion technique where the virion envelope fuses with the cellular membrane of the host cell and directly releases the capsid containing the RNA genome and replicating enzymes into the cytoplasm of the host cell. HIV utilizes enzymes created by its own genome and enzymes native to the T-Helper cell to generate the proteins it requires to construct copies of HIV. Once the appropriate copies of the RNA, nucleocapsid protein, integrase, protease, reverse transcriptase enzyme, capsid proteins, matrix protein and external probes have been manufactured and collected together, the capsid carrying the RNA genetic payload is enveloped by the matrix protein and pushes through the host cell's plasma membrane in a process called budding. The HIV copy becomes encapsulated in an envelope comprised of a lipid bilayer as it separates from the host cell and becomes an independent entity termed a virion. Probes stick out through the lipid bilayer envelope of the virion to seek the receptors located on an appropriate host cell.

To produce copies of the SMART-Fas/FasL Virus a T-Helper cell can be utilized, bacteria with a lipid bilayer membrane could be utilized, hybrid cells (combination of animal cell, plant cell or bacteria) could be utilized or other appropriate host cell could be utilized. The technology to manufacture viruses carrying a therapeutic DNA gene has already been worked out and implemented. The process to generate a medically therapeutic virus to target a particular type of cell is a matter of placing inside a host cell the appropriate genetic instructions and enzymes to facilitate the host cell to manufacture the intended ‘medically therapeutic’ virus.

In the case of the SMART-Fas/FasL Virus, the construction of the medically therapeutic copies of the SMART-FasL Virus include the appropriate instructions and biologic machinery necessary to generate the capsid proteins, matrix proteins, external cell-surface receptors Fas and FasL, any filling material to be placed inside the capsid and any cell instruction proteins necessary to stimulate and manage the budding process. The Fas cell-surface receptor is a naturally occurring cell-surface receptor on T-Helper cells. The genetic instruction code for manufacturing the FasL receptor and fixing it on the lipid bilayer as a receptor is available and carried in the HIV genome. The size of the capsid is dependent upon the quantity of capsid proteins used to construct the capsid. For descriptive purposes the use of the term ‘capsid’ is interchangeable with the term ‘capsid shell’. Different naturally occurring viruses are constructed with a different size capsid depending upon the number and arrangement of capsid proteins utilized to construct a virus's capsid. The diameter of the SMART-Fas/FasL Virus is in part dependent upon the number and arrangement of capsid proteins used to construct the capsid. A genetic payload to act as a filler, incapable of stimulating a disease state in any form of life, may be required to fill the inside the capsid of some of the larger diameter SMART-Fas/FasL Viruses in order to support the successful construction of a particular size of SMART-Fas/FasL Virus and to facilitate the SMART-Fas/FasL Virus remaining sturdy enough and thus intact once released into the environment to enable it to successfully carry out its intended function.

The size of the SMART-Fas/FasL Virus is between 7 nm in thickness (the diameter of HIV is approximately 50 to 150 nm) to a diameter in the order of the size of a naturally occurring noninfected T-Helper cell, to a diameter of one meter. The size of the SMART-Fas/FasL Virus is variable due to the range of applications for which the SMART-Fas/FasL Virus is intended to be utilized. The SMART-Fas/FasL Virus may take on the shape of simply a relatively flat sheet of varying sizes which may be folded into various configurations, to spherical structures of varying sizes to irregularly shaped convoluted structures of varying sizes. The unique principle of intent is to engage infected T-Helper cells with Fas cell-surface receptors and FasL cell-surface receptors in whatever manner might terminate T-Helper cells infected with the Human Immunodeficiency Virus.

### DRAWING

None.

What is claimed:

1. A medical device comprised of a lipid bilayer envelope, represented as an independent sheet or a covering, which affixed to the said envelope are two or more different types of cell-surface receptors intended to attract and functionally engage a target leukocyte with the intention of terminating the target leukocyte for medical treatment purposes.

2. A medical device comprised of a lipid bilayer envelope, represented as an independent sheet or a covering, which affixed to the said envelope are one or more Fas cell-surface receptors and one or more FasL cell-surface receptors for medical treatment purposes.

3. A medical device comprised of a lipid bilayer envelope, represented as an independent sheet or a covering, which affixed to the said envelope are two or more different types of cell-surface receptors intended to attract and functionally engage an infected eukaryote with the intention of terminating the infected eukaryote for medical treatment purposes.

4. A medical device comprised of a lipid bilayer envelope, represented as an independent sheet or a covering, which affixed to the said envelope are two or more different types of cell-surface receptors intended to attract and functionally engage target cell-surface receptors located on the surface of a T-Helper cell infected with Human Immunodeficiency Virus with the intention of terminating the infected T-Helper cell for the purpose of neutralizing the infectious threat of the Human Immunodeficiency Virus.

5. A medical device comprised of a lipid bilayer envelope, represented as an independent sheet or a covering, which affixed to the said envelope are one or more Fas cell-surface receptors and one or more FasL cell-surface receptors intended to attract and functionally engage target cell-surface receptors located on the surface of one or more T-Helper cells infected with Human Immunodeficiency Virus, where the Fas cell-surface receptor is present on the envelope of the medical device in a fashion that the Fas cell-surface receptor will be physically engaged by an infected T-Helper cell before an FasL receptor can be physically engaged by the same infected T-Helper cell, with the intention of initiating apoptosis in T-Helper cells infected with the Human Immunodeficiency Virus in order to terminate these same T-Helper cells infected with the Human Immunodeficiency Virus for the purpose of neutralizing the infectious threat of the Human Immunodeficiency Virus.

6. A medical device comprised of a capsid shell constructed of repeating capsid proteins, this capsid shell encapsulated by matrix proteins, further encapsulated by an exterior lipid bilayer envelope, which affixed to the outer envelope are two or more different types of exterior cell-surface receptors intended to attract and functionally engage target cell-surface receptors located on the surface of a T-Helper cell infected with Human Immunodeficiency Virus with the intention of initiating apoptosis in T-Helper cells infected with the Human Immunodeficiency Virus in order to terminate these same T-Helper cells infected with the Human Immunodeficiency Virus for the purpose of neutralizing the infectious threat of the Human Immunodeficiency Virus.

7. A medical device comprised of a capsid shell constructed of repeating capsid proteins, this capsid shell encapsulated by matrix proteins, further encapsulated by an exterior lipid bilayer envelope, which affixed to the outer envelope are one or more exterior surface cell-receptors known as Fas, as found on naturally occurring T-Helper cells, and one or more exterior cell-surface receptors known as FasL, as found on T-Helper cells infected with the Human Immunodeficiency Virus.

8. A medical device comprised of a capsid shell constructed of repeating capsid proteins, this capsid shell encapsulated by matrix proteins, further encapsulated by an exterior lipid bilayer envelope, which affixed to the outer envelope are at least two different exterior cell-surface receptors each intended to functionally engage a cell-surface receptor located on the surface of T-Helper cell infected with Human Immunodeficiency Virus expressing one or more FasL cell-surface receptors, with the intention of initiating apoptosis in T-Helper cells infected with the Human Immunodeficiency Virus in order to terminate these same T-Helper cells infected with the Human Immunodeficiency Virus for the purpose of neutralizing the infectious threat of the Human Immunodeficiency Virus.

9. A medical device comprised of a capsid shell constructed of repeating capsid proteins, this capsid shell encapsulated by matrix proteins, further encapsulated by an exterior lipid bilayer envelope, all three elements similar in construct to the materials and design of the naturally occurring Human Immunodeficiency Virus with the exterior envelope having fixed to its exterior cell-surface receptors constructed in similar physical form and dimensions as generally known and recognized by the medical scientific community as the Fas cell-surface receptor as generally found on a naturally occurring human T-Helper cell and the FasL cell-surface receptor generally found on a T-Helper cell infected with HIV in a manner and design that the medical device's Fas cell-surface receptors will attract and functionally engage one or more FasL cell-surface receptors on a T-Helper cell infected with the Human Immunodeficiency Virus and one or more FasL cell-surface receptors on the medical device will attract and functionally engage one or more Fas cell-surface receptors on the same said HIV infected T-Helper cell with the intention of initiating apoptosis in T-Helper cells infected with the Human Immunodeficiency Virus in order to terminate these same T-Helper cells infected with the Human Immunodeficiency Virus for the purpose of neutralizing the infectious threat of the Human Immunodeficiency Virus.

10. A medical device comprised of a capsid shell constructed of repeating capsid proteins, this capsid shell encapsulated by matrix proteins, further encapsulated by an exterior lipid bilayer envelope, all three elements similar in construct to the materials and design of the naturally occurring Human Immunodeficiency Virus with the exterior envelope having fixed to its exterior cell-surface receptors constructed in the same physical form and dimensions as generally known and recognized by the medical scientific community as the Fas cell-surface receptor as generally found on naturally occurring human T-Helper cells and the FasL cell-surface receptor generally found on a T-Helper cell infected with HIV, in a manner and design that a Fas cell-surface receptor located on the exterior of the medical device will attract and functionally engage a FasL cell-surface receptor on a T-Helper cell infected with HIV and a FasL cell-surface receptor on the medical device will attract and functionally engage a Fas cell-surface receptor on the same said HIV infected T-Helper cell, where the Fas cell-surface receptors are present on the outer envelope of the medical device in a fashion that a Fas cell-surface receptor will be physically engaged by an infected T-Helper cell before a FasL receptor can be physically engaged by the same infected T-Helper cell, with the intention of initiating apoptosis in T-Helper cells infected with the Human Immunodeficiency Virus in order to terminate these same T-Helper cells infected with the Human Immunodeficiency Virus for the purpose of neutralizing the infectious threat of the Human Immunodeficiency Virus.

11. A medical device comprised of a capsid shell constructed of repeating capsid proteins, this capsid shell encapsulated by matrix proteins which by this construct determines the size of the device, which is further encapsulated by an exterior lipid bilayer envelope, all three elements similar in construct to the materials and design of the naturally occurring Human Immunodeficiency Virus with the exterior envelope having fixed to its exterior cell-surface receptors constructed in the same physical form and dimensions as generally known and recognized by the medical scientific community as the Fas cell-surface receptor as generally found on naturally occurring human T-Helper cells and the FasL cell-surface receptor generally found on a T-Helper cell infected with HIV, in a manner and design that a Fas cell-surface receptor located on the exterior of the medical device will attract and functionally engage a FasL cell-surface receptor on a T-Helper cell infected with HIV and a FasL cell-surface receptor on the medical device will attract and functionally engage a Fas cell-surface receptor on the same said HIV infected T-Helper cell, where the Fas cell-surface receptors are present on the outer envelope of the medical device in a fashion that a Fas cell-surface receptor will be physically engaged by an infected T-Helper cell before a FasL receptor can be physically engaged by the same infected T-Helper cell, with the intention of initiating apoptosis in T-Helper cells infected with the Human Immunodeficiency Virus in order to terminate these same T-Helper cells infected with the Human Immunodeficiency Virus for the purpose of neutralizing the infectious threat of the Human Immunodeficiency Virus.

12. A medical device of an overall size that ranges from a thickness of 7 nm to a size in the order of a T-Helper cell approximately 3500 nm in diameter, comprised of a capsid shell constructed of repeating capsid proteins, this capsid shell encapsulated by matrix proteins which by this construct determines the size of the device, which is further encapsulated by an exterior lipid bilayer envelope, all three elements similar in construct to the materials and design of the naturally occurring Human Immunodeficiency Virus with the exterior envelope having fixed to its exterior cell-surface receptors constructed in the same physical form and dimensions as generally known and recognized by the medical scientific community as the Fas cell-surface receptor as generally found on naturally occurring human T-Helper cells and the FasL cell-surface receptor generally found on a T-Helper cell infected with HIV, in a manner and design that a Fas cell-surface receptor located on the exterior of the medical device will attract and functionally engage a FasL cell-surface receptor on a T-Helper cell infected with HIV and a FasL cell-surface receptor on the medical device will attract and functionally engage a Fas cell-surface receptor on the same said HIV infected T-Helper cell, where the Fas cell-surface receptors are present on the outer envelope of the medical device in a fashion that a Fas cell-surface receptor will be physically engaged by an infected T-Helper cell before a FasL receptor can be physically engaged by the same infected T-Helper cell, with the intention of initiating apoptosis in T-Helper cells infected with the Human Immunodeficiency Virus in order to terminate these same T-Helper cells infected with the Human Immunodeficiency Virus for the purpose of neutralizing the infectious threat of the Human Immunodeficiency Virus.

13. A medical device of an overall size that ranges from a thickness of 7 nm to a size in the order of a T-Helper cell approximately 3500 nm in diameter, comprised of a capsid shell constructed of repeating capsid proteins, this capsid shell encapsulated by matrix proteins which by this construct determines the size of the device, which is further encapsulated by an exterior lipid bilayer envelope, all three elements similar in construct to the materials and design of the naturally occurring Human Immunodeficiency Virus with the exterior envelope having fixed to its exterior cell-surface receptors constructed in the same physical form and dimensions as generally known and recognized by the medical scientific community as the Fas cell-surface receptor as generally found on naturally occurring human T-Helper cells and the FasL cell-surface receptor generally found on a T-Helper cell infected with HIV, in a manner and design that a Fas cell-surface receptor located on the exterior of the medical device will attract and functionally engage a FasL cell-surface receptor on a T-Helper cell infected with HIV and a FasL cell-surface receptor on the medical device will attract and functionally engage a Fas cell-surface receptor on the same said HIV infected T-Helper cell, where the Fas cell-surface receptors are present on the outer envelope of the medical device in a fashion that a Fas cell-surface receptor will be physically engaged by an infected T-Helper cell before a FasL receptor can be physically engaged by the same infected T-Helper cell, with the intention of initiating apoptosis in T-Helper cells infected with the Human Immunodeficiency Virus in order to terminate these same T-Helper cells infected with the Human Immunodeficiency Virus for the purpose of neutralizing the infectious threat of the Human Immunodeficiency Virus, which the medical device carries a genetic payload to act as a filler, incapable of stimulating a disease state in any form of life, required to fill the inside of the capsid of the medical device in order to support the successful construction of a particular size of the medical device and to facilitate the medical device remaining sturdy enough during production and to insure the medical device will remain intact and functional once administered as a treatment into an environment where HIV infected T-Helper cells may exist.

14. A medical device of an overall size that ranges from a thickness of 7 nm to a size to a diameter of one meter, comprised of a capsid shell constructed of repeating capsid proteins, this capsid shell encapsulated by matrix proteins which by this construct determines the size of the device, which is further encapsulated by an exterior lipid bilayer envelope, all three elements similar in construct to the materials and design of the naturally occurring Human Immunodeficiency Virus with the exterior envelope having fixed to its exterior cell-surface receptors constructed in the same physical form and dimensions as generally known and recognized by the medical scientific community as the Fas cell-surface receptor as generally found on naturally occurring human T-Helper cells and the FasL cell-surface receptor generally found on a T-Helper cell infected with HIV, in a manner and design that a Fas cell-surface receptor located on the exterior of the medical device will attract and functionally engage a FasL cell-surface receptor on a T-Helper cell infected with HIV and a FasL cell-surface receptor on the medical device will attract and functionally engage a Fas cell-surface receptor on the same said HIV infected T-Helper cell, with the intention of initiating apoptosis in T-Helper cells infected with the Human Immunodeficiency Virus in order to terminate these same T-Helper cells infected with the Human Immunodeficiency Virus for the purpose of neutralizing the infectious threat of the Human Immunodeficiency Virus, which the medical device carries a genetic payload to act as a filler, incapable of stimulating a disease state in any form of life, required to fill the inside of the capsid of the medical device in order to support the successful construction of a particular size of the medical device and to facilitate the medical device remaining sturdy enough during production and to insure the medical device will remain intact and functional once administered as a treatment into an environment where HIV infected T-Helper cells may exist.

15. A medical device of an overall size that ranges from a thickness of 7 nm to a size to a diameter of one meter, comprised of a capsid shell constructed of repeating capsid proteins, this capsid shell encapsulated by matrix proteins which by this construct determines the size of the device, which is further encapsulated by an exterior lipid bilayer envelope, all three elements similar in construct to the materials and design of the naturally occurring Human Immunodeficiency Virus with the exterior envelope having fixed to its exterior cell-surface receptors constructed in the same physical form and dimensions as generally known and recognized by the medical scientific community as the Fas cell-surface receptor as generally found on naturally occurring human T-Helper cells and the FasL cell-surface receptor generally found on a T-Helper cell infected with HIV, in a manner and design that a Fas cell-surface receptor located on the exterior of the medical device will attract and functionally engage a FasL cell-surface receptor on a T-Helper cell infected with HIV and a FasL cell-surface receptor on the medical device will attract and functionally engage a Fas cell-surface receptor on the same said HIV infected T-Helper cell, where the Fas cell-surface receptors are present on the outer envelope of the medical device in a fashion that a Fas cell-surface receptor will be physically engaged by an infected T-Helper cell before a FasL receptor can be physically engaged by the same infected T-Helper cell, with the intention of initiating apoptosis in T-Helper cells infected with the Human Immunodeficiency Virus in order to terminate these same T-Helper cells infected with the Human Immunodeficiency Virus for the purpose of neutralizing the infectious threat of the Human Immunodeficiency Virus, which the medical device carries a genetic payload to act as a filler, incapable of stimulating a disease state in any life form, required to fill the inside of the capsid of the medical device in order to support the successful construction of a particular size of the medical device and to facilitate the medical device remaining sturdy enough during production and to insure the medical device will remain intact and functional once administered as a treatment into an environment where HIV infected T-Helper cells may exist.
